(19) United States
(12) Patent Application Publication
(10) Pub. No.: US 2009/0299233 A1
(43) Pub. Date: Dec. 3, 2009

(54) Device for Measuring Functional Reach

(75) Inventors: Ching-Yi Wang, Taichung City (TW); Yung-Wen Tang, Taichung City (TW)

Correspondence Address: WPAT, PC;INTELLECTUAL PROPERTY ATTORNEYS, 2030 MAIN STREET, SUITE 1300, IRVINE, CA, 92614, US

(73) Assignee: CHUNG SHAN MEDICAL UNIVERSITY, Taichung City (TW)

(21) Appl. No.: 12/435,106

(22) Filed: May 4, 2009

(30) Foreign Application Priority Data
May 27, 2008 (TW) 097209217

(51) Int. Cl. A61B 5/103; G01B 1/00

(52) U.S. Cl. 600/595; 33/512

(57) ABSTRACT

A device for measuring functional reach is disclosed, which comprises three parts: a support, one end thereof being attached to an object; a ruler, one end thereof being connected to the support; and a vernier, being connected to the ruler and moving on the ruler forward and backward. The invention is used for a functional evaluation test for measuring balance.

### FIELD OF THE INVENTION

The present invention relates to a device for measuring functional reach.

### DESCRIPTION OF PRIOR ART

A functional evaluation test is used for measuring balance at a clinic these days, in which a ruler is used for measuring a distance of stretching forward, with the aid of reading graduation on the ruler. Besides, since people to be examined may have short duration for keeping their arms at a horizontal pose needed in the test or tend to shake their hands up and down because of their infirmity, weakness, and poor balance, an examining people not only has to pay attention on keeping them safe during the test, but also has to read the graduation on the ruler. Thus, the accuracy of test data will be affected, resulting in a possible reading error. It is desired to provide a device capable of facilitating operations easily by an examining person, reducing the reading error, and increasing the reading accuracy, while capable of providing an examination result objectively but not subjectively, so as to help medical staffs to provide the people to be examined with suitable treatments.

### BRIEF DESCRIPTION OF THE DRAWINGS

FIG. 1 shows a device for measuring functional reach according to the present invention.

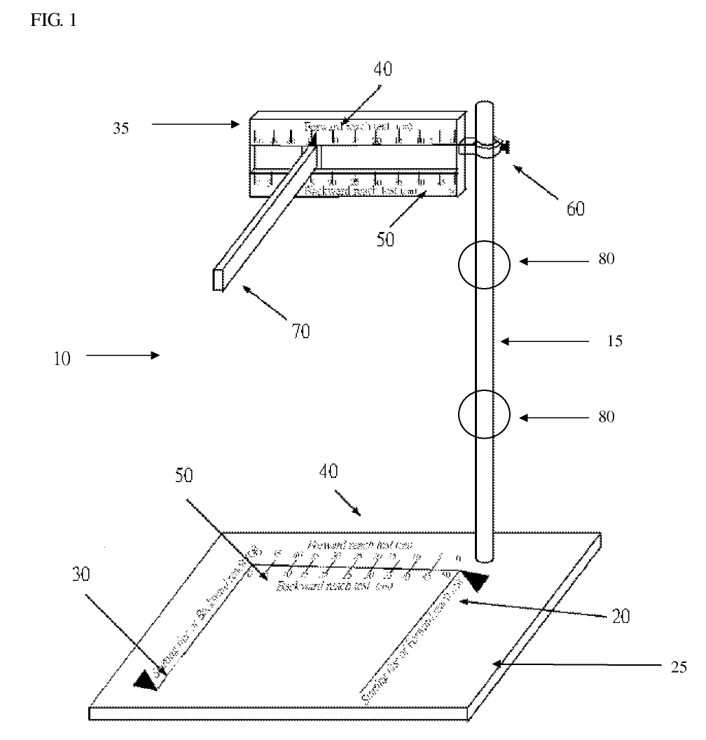

### SUMMARY OF THE INVENTION

The present invention provides a device for measuring functional reach, comprising:
a) a support, one end thereof being attached to an object;
b) a ruler, one end thereof being connected to the support; and
c) a vernier, being connected to the ruler and moving on the ruler forward and backward.

### DETAILED DESCRIPTION OF THE INVENTION

A device 10 of the present invention comprises three parts: (1) a support, one end thereof being attached with an object; (2) a ruler 35, one end thereof being connected to the support; and (3) a vernier 70, being connected to the ruler and moving on the ruler forward and backward.

The support of the invention is a supporting rod 15, which may be attached to an object such as a tripod or flat plate 25, and so on. To be attached to the tripod, the supporting rod is accommodated to moving, and to be attached to the flat plate, the supporting rod has to be fixed with a tenon. A testing start line 20 for stretching forward (stretching right) or a testing start line 30 for stretching backward (stretching left) is provided on the flat plate, wherein a shifting distance 40 for stretching forward (stretching right) and a further shifting distance 50 for stretching backward (stretching left) are respectively provided between the testing start line 20 for stretching forward (stretching right) and the testing start line 30 for stretching backward (stretching left). The testing start line on the flat plat is not necessary because the distance between beginning and ending is the result. The ruler is connected to the supporting rod by a movable buckle 60, a vernier is protruded on a surface of the ruler, and an electronic or non-electronic shifting detector is provided on the vernier for measuring a moving distance of the vernier. Further, a junction is further provided on the supporting rod, or the movable buckle 60 is replaced with a junction, for measuring extendedly or contractedly and for storing.

The invention can be used for concurrently measuring functional stretching distances in four directions, i.e. functional stretching forward, backward, right, and left for a person to be examined.

The invention is especially useful to measure the balance ability of a person to be examined in rehabilitation (orthopedics, neuropathology, cardiopulmonary, pediatric, kinesiology and geriatrics), family medicine, internal medicine and surgery, kinesiology, occupational therapy, clinic, long term care institutions, or research unit.

### EXAMPLE

The examples below are non-limiting and are merely representative of various aspects and features of the present invention.

### Example 1

### Steps for Preparing the Invention

As showed in the device 10 mainly comprised a supporting rod 15, a flat plate 25, a ruler 35, and a vernier 70. There were a testing start line 20 for stretching forward (stretching right) and a testing start line 30 for stretching backward (stretching left) on the flat plate.

On the ruler 35, the shifting distances corresponding to those sensed by an electronic or non-electronic shifting detector for detecting the shifting distances, i.e. the corresponding distance 40 of stretching forward (stretching right) and the corresponding distance 50 of stretching backward (stretching left) were labeled. The supporting rod was connected to the ruler by a movable buckle 60. The position of the ruler on the supporting rod could be adjusted, through the movable buckle, to the level of the horizontally stretched arm of the individual to be tested. The flat plate could be replaced with a tripod and two tripod junctions 80 could be provided on the supporting rod, thereby proceeding with measurements in response to a taller person to be examined and also for storing. The movable buckle could be replaced with a junction for the ruler to facilitate measuring and storing.

### Example 2

### Operating Steps

The operating steps of the functional stretching forward test:

1. To activate the detectors. (This step was only needed if an electronic detector was used.)

2. To help the person to be examined to stand at the testing start line 20 for stretching forward, face the vernier, lifted his or her arm forward horizontally, and made a fist, while adjusting the height of the ruler and setting the touching place on the ruler as the start point of the shifting test.

3. To zero the detector and define the start point of shifting.

4. The person to be examined stretches forward with his or her full power to push and move the vernier for ensuring the electronic detector to have detected the shift of the ruler. After reaching and stopping at the farthest shift distance in a balance state maintained by the person to be examined, the functional stretching forward test was completed and a stretching distance could be read from the electronic or non-electronic detector on the ruler.

If an electronic detector was used, the distance reached could be read out directly on the detector at the end of the test. If a non-electronic detector was used, then it would be the ruler with graduation on it, the difference between the graduation at starting position and the graduation at end position would be the distance reached by the individual.

The operating steps of functional reach (stretching backward) test were similar to those of the functional reach (stretching forward) test, with the differences in step 2, the person to be examined standing at the testing start line 30 for stretching backward (stretching left), facing the vernier and lifting his or her arm forward horizontally, and in step 4, the person to be examined stretching backward. The operating steps of functional stretching left test were similar to those of the functional stretching forward test, with the differences in step 2, the person to be examined standing at the testing start line 30, with back facing the ruler, for stretching left, and lifting his or her left arm up horizontally, and in step 4, the person to be examined stretching left.

The operating steps of functional stretching right test were similar to those of the functional stretching forward test, with the differences in step 2, the person to be examined standing at the testing start line 20 for stretching forward (stretching right), with back facing the vernier and lifting his or her right arm up horizontally, and in step 4, the person to be examined stretching right.

Although the present invention has been explained in relation to its preferred embodiments, it is to be understood that many other possible modifications and variations can be made without departing from the scope of the invention as hereinafter claimed.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The embryos, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A device for measuring functional reach, comprising:
a) a support, one end thereof being attached to an object;
b) a ruler, one end thereof being connected to the support; and
c) a vernier, being connected to the ruler and moving on the ruler forward and backward.

2. The device of claim 1, wherein the support is a supporting rod.

3. The device of claim 1, wherein the object is a tripod or a flat plate.

4. The device of claim 3, wherein the tripod is accommodated to moving.

5. The device of claim 3, wherein the flat plate is attached to the supporting rod with a tenon.

6. The device of claim 5, wherein a testing start line for stretching forward (stretching right) or a testing start line for stretching backward (stretching left) is provided on the flat plate.

7. The device of claim 6, wherein a shifting distance for stretching forward (stretching right) and a further shifting distance for stretching backward (stretching left) are respectively provided the potential distance for reaching during the stretching forward (stretching right) and the stretching backward (stretching left).

8. The device of claim 2, wherein the ruler is connected to the supporting rod by a movable buckle.

9. The device of claim 1, wherein the vernier protrudes on a surface of the ruler.

10. The device of claim 1, wherein the ruler is provided with an electronic or non-electronic shifting detector for measuring a moving distance of the vernier.

11. The device of claim 2, wherein a junction is further provided on the supporting rod for measuring extendedly or contractedly and for storing.

12. The device of claim 8, wherein the movable buckle is replaced with a junction for measuring extendedly or contractedly and for storing.

13. The device of claim 5, wherein the start lines are not necessary if a tripod is used to replace the flat plate.

14. The device of claim 8, wherein the ruler can be moved to parallel to the horizontally outstretched arm through the movable buckle.
